(19) United States
(12) Patent Application Publication
(10) Pub. No.: US 2015/0136638 A1
(43) Pub. Date: May 21, 2015

(54) METHOD FOR PREPARING A SYRUPY PRODUCT COMPRISING VITAMINS

(71) Applicants: Franck Eymard, Talant (FR); Pierre Gabiot, Dijon (FR); Romain Brenon, Gissey Sur Ouche (FR); Michel Lamoise, Bessey Les Citeaux (FR)

(72) Inventors: Franck Eymard, Talant (FR); Pierre Gabiot, Dijon (FR); Romain Brenon, Gissey Sur Ouche (FR); Michel Lamoise, Bessey Les Citeaux (FR)

(73) Assignee: LaboratoiresUrgo, Chenove (FR)

(21) Appl. No.: 14/406,530

(22) PCT Filed: Jan. 29, 2013
(86) PCT No.: PCT/FR2013/050179

§ 371 Date: Dec. 8, 2014

(30) Foreign Application Priority Data
Jan. 30, 2012 (FR) PCT/FR12/50197

(51) Int. Cl. A61K 31/4415; A61K 31/375; A61K 31/525; A61K 31/197; A61K 47/36; A61K 31/355; A61K 47/26; A61K 31/714; A61K 31/593; A61K 31/519; A61K 31/455; A61K 31/07

(52) U.S. Cl. CPC A61K 31/4415; A61K 31/455; A61K 31/375; A61K 31/525; A61K 31/197; A61K 31/07; A61K 31/355; A61K 47/26; A61K 31/714; A61K 31/593; A61K 31/519; A61K 47/36
USPC 206/525; 514/251; 514/52; 514/168

(57) ABSTRACT

The present invention relates to a method for preparing a syrupy product comprising vitamins, said method comprising the following steps: a) inerting all the apparatuses implemented in the method b) inserting sugar syrup into said apparatuses and adding vitamins under inert atmosphere; c) adding dehydrated and deoxygenated glucose under inert atmosphere; d) deoxygenating the syrupy product formed in this way; e) packaging the syrupy product under inert atmosphere.

The present invention relates to a process for preparing a syrupy product comprising vitamins. Most, or even all, of the steps of the process are carried out under an inert atmosphere. Furthermore, the syrupy product is completely deoxygenated before it is packaged in order to have a nonexistent dissolved oxygen content in the syrupy product, thereby making it possible to avoid degradation of the vitamins during the preparation of the syrupy product, during its storage once packaged, and even after a first consumption of the product by the user.

A healthy balanced diet is supposed to make it possible to meet the recommended daily intakes (RDI) in terms of vitamins and minerals. However, modern eating habits can sometimes cause certain dietary deficiencies.

Vitamins, in a dose ranging from a microgram to several milligrams per day, are necessary for the metabolism of living organisms and in particular that of human beings. However, the organism generally cannot synthesize them in the amounts recommended for meeting daily needs. In human beings, only three vitamins are synthesized by intestinal bacteria: vitamins K, B8 and B12.

An insufficient intake or a lack of vitamin causes, respectively, hypovitaminosis or avitaminosis which can be the cause of various diseases (scurvy, beriberi, rickets, etc).

Furthermore, vitamins play an essential role in child growth and development. Thus, fat-soluble vitamins (such as vitamins A, E or K), and more preferentially vitamin D, prove to be essential to children and adolescents.

Vitamin D plays an essential role in the intestinal absorption of calcium and mineralization of the skeleton. This vitamin D requirement may be necessary for children, especially when the latter live in regions where there is a low amount of sunshine, even more so during winter.

With regard to water-soluble vitamins, vitamins C and B1 are particularly important for both young children and adolescents or adults.

It is therefore often necessary to provide, by means of food supplements, various combinations of vitamins in a concentrated form so as to supplement the diet of an individual.

However, the formulation of vitamin-containing food supplements is challenging in view of the problem of the great instability of vitamins with respect to oxygen, temperature, pH or light.

Vitamin-containing food supplements are thus most commonly formulated in the form of capsules so that the vitamins are not in direct contact with ambient oxygen, such as the capsules sold by the company Nycomed® under the name Dynatonic®.

It is, however, desirable to propose vitamin formulations in liquid form, in particular in the form of a syrupy product, which is easier to administer, especially in the case of children.

Syrupy products comprising vitamins have already been proposed. However, in order to compensate for the degradation of the vitamins in contact with oxygen, it has up until now been necessary to introduce the vitamins in an initial amount that is greater than the one that is actually present in the final product, i.e. to use the technique of initial overdose of vitamin products in order to obtain a syrupy product having an individual and/or overall vitamin ratio close to 100%.

For the purposes of the present invention, the term “individual vitamin ratio” is intended to mean the ratio of the amount of a vitamin introduced to the amount of said vitamin found at the end of the process. It is calculated individually for each of the vitamins introduced in the process according to the invention.

For the purposes of the present invention, the term “overall vitamin ratio” is intended to mean the ratio of the introduced amount of all the vitamins to the amount of said vitamins found at the end of the process. This ratio is calculated for all of the vitamins introduced in the process according to the invention.

Furthermore, the vitamin degradation has a tendency to continue during the period of storage of the syrupy product in its packaging. The overdose of the vitamins and the limited shelf life of the product generate a considerable additional expenditure for vitamin products in liquid form, thereby making them not very cost efficient in industrial terms.

Patent EP 1 320 356 describes a process for preparing a syrup comprising vitamins which has a long shelf life. The syrup is in the form of an oil-in-water emulsion prepared by mixing an aqueous phase containing water-soluble vitamins, a gelling agent and a thickener, with an oil phase containing the fat-soluble vitamins and a surfactant. The fat-soluble vitamins are inside the oil particles dispersed in the aqueous continuous phase and are thus protected against oxidation by the air. When the syrup also contains water-soluble vitamins, they are inside water droplets included in the oil particles dispersed in the aqueous phase, which results in a water-in-oil-in-water emulsion which can pose storage stability problems during the manufacturing process, but also after packaging of said product, and even more so after a first use of the product by the consumer. In this patent, the problem of vitamin losses as soon as the syrup manufacturing process is initiated is solved only by means of an overdose of vitamins at the beginning of the process, in order to obtain an overall vitamin ratio close to 100% at the end of the process. Thus, no solution to the problem of overdose of unstable raw materials in a process for manufacturing a syrupy product of pharmaceutical quality is provided by this document.

There is therefore a real need for the development of a novel process for manufacturing a syrupy product which comprises vitamins and which exhibits good storage stability without overdose of the amount of vitamins when they are introduced into the process and which makes it possible to obtain an optimum individual and/or overall vitamin ratio at the end of the process.

The applicant has found that it is possible to obtain a simple and economical process for manufacturing a syrupy product which makes it possible to optimize the amount of vitamins introduced as raw materials by preventing but also avoiding their degradation during the preparation and in the packaged final product.

An object of the invention is therefore a process for preparing a syrupy product comprising vitamins, in a pharmaceutically acceptable medium, said process comprising the following steps:
a) inerting all the equipment used in the process;
b) introducing, into said equipment, a sugar syrup and introducing the vitamins under an inert atmosphere;
c) adding dehydrated and deoxygenated glucose under an inert atmosphere;
d) deoxygenating the syrupy product thus formed;
e) packaging the syrupy product under an inert atmosphere.

According to another aspect, an object of the invention is also a process for preparing a syrupy product comprising vitamins, in a pharmaceutically acceptable medium, said process comprising the following steps:
a) inerting all the equipment used in the process;
b) introducing, into said equipment, a sugar syrup and/or a sugar derivative and introducing the vitamins under an inert atmosphere;
c) optionally, adding dehydrated and deoxygenated glucose under an inert atmosphere;
d) deoxygenating the syrupy product thus formed;
e) packaging the syrupy product under an inert atmosphere.

For the purposes of the present invention, the term “pharmaceutically acceptable medium” is intended to mean a medium which is compatible with oral administration of the syrupy product. The pharmaceutically acceptable medium may in particular comprise water. According to one preferred embodiment, the pharmaceutically acceptable medium does not comprise any solvent other than water, purified and deoxygenated beforehand.

For the purposes of the present invention, the term “sugar syrup” is intended to mean a solution of sugar in demineralized water. The term “sugar” is intended to mean any monosaccharide, such as, in particular, glucose, dextrose, fructose, galactose, or mannose, or any disaccharide, such as saccharose (also known as sucrose), lactose or maltose, conferring a sweet taste on the food into which they are introduced. In the sugar syrup used in the process according to the invention, the amount of sugar, in particular of saccharose, can in particular range from 65% to 70% by weight relative to the total weight of the sugar syrup, preferably from 66.5% to 67.5% and more preferentially be about 67%.

The term “sugar derivative” is intended to mean any synthetic or natural substance of which the sweetening power is comparable to or higher than that of saccharose. The sugar derivative can thus be a sweetener which, compared with sugar, exhibits a low carie-causing capacity, a low calorific value and a benefit to health for diabetic individuals.

For the purposes of the present application, the term “syrupy product” is intended to mean a product based on sugar syrup and/or on sugar derivative, it being possible, moreover, for said syrupy product to comprise any other compound, such as, in particular, vitamins. The syrupy product in particular has a viscosity higher than that of water, but remains fluid and can in particular flow under its own weight.

For the purposes of the present invention, the term “vitamins” is intended to mean organic molecules required for the growth and correct functioning of the body of living beings. Vitamins are conventionally divided up into two categories according to their solubility: water-soluble vitamins and fat-soluble vitamins.

Examples of water-soluble vitamins present in the syrupy product prepared according to the invention are in particular: vitamin B1 (thiamine), vitamin B2 (riboflavin), vitamin B3 (nicotinic acid), also known as vitamin PP (Pellagra Preventive), vitamin B5 (pantothenic acid), vitamin B6 (pyridoxine), vitamin B8 or H (biotin), vitamin B9 (folic acid), vitamin B12 (cobalamin) and vitamin C (ascorbic acid).

Examples of fat-soluble vitamins present in the syrupy product prepared according to the invention are in particular: vitamin A (retinol), vitamin D3 (cholecalciferol), vitamin E (mixture of tocopherols and tocotrienols) and vitamin K (phylloquinone).

For the purposes of the present invention, the term “dehydrated glucose” is intended to mean a glucose powder having a water content of less than 10%, and preferably less than 5%.

### FIGURE

FIG. 1 illustrates, diagrammatically, the process according to the present invention.

### INERTING OF EQUIPMENT

The process according to the invention comprises a step a) of initial inerting of all the equipment used in the preparation of the syrupy product.

For the purposes of the present application, the term “inerting” is intended to mean replacing the air contained in a volume with an inert gas.

For the purposes of the present application, the term “equipment” is intended to mean all the mechanical devices used in carrying out the process according to the present invention, such as, in particular, the various tanks, the powder-transferring device, the static mixer, the hopper located upstream of the packaging device, the packaging device and the pipes.

It should be noted that, subsequently, the inerting of the equipment can preferably be maintained by a continuous bubbling carried out in each piece of said equipment.

The step of inerting the equipment used in the process according to the invention, such as the tanks, the powder-transferring device, the static mixer, the packaging device and the pipes, makes it possible in particular to avoid degradation of the vitamins during the process.

The inerting can in particular be carried out by injecting an overpressure of inert gas into the tanks, the powder-transferring device, the static mixer, the packaging device and the pipes for a period sufficient to obtain an acceptable oxygen content.

For the purposes of the present invention, the term “acceptable oxygen content” is intended to mean an oxygen content of between 0 and 4% by volume of oxygen per volume of product, preferably between 0 and 2% by volume and more preferentially less than 0.1% by volume.

The oxygen content can be controlled in particular in the tanks or the pipes via a probe placed in said tanks and pipes.

In particular, the oxygen content is measured with a Mettler InPro 6850i probe, according to the standard parameters provided by the manufacturer.

The injection of inert gas into the equipment, in particular into the tanks and the pipes, can in particular be carried out at a pressure ranging from 0.1 to 12 bar, preferably from 0.2 to 5 bar and more preferentially still of about 0.45 bar.

The injection of inert gas into the equipment, in particular into the tanks and the pipes, can in particular be carried out for a period of time ranging from 100 to 1000 seconds, preferably from 200 to 800 seconds and more preferentially from 300 to 600 seconds.

The inert gas, free of oxygen, that is injected can in particular be nitrogen or carbon dioxide, preferably the inert gas is nitrogen.

Introduction of the Sugar Syrup and/or Sugar Derivative and Vitamins

The process according to the invention comprises a step b) of introducing the sugar syrup and/or sugar derivative and introducing the vitamins. According to one preferred embodiment, the sugar syrup is a saccharose-based syrup.

According to another preferred embodiment, the sugar derivative can be chosen from the nonlimiting list comprising, in particular, dextrin-based soluble fibers and polyols.

For the purposes of the present invention, the expression “dextrin-based soluble fibers” is intended to mean any partially hydrolyzed residue which results from a process of heating, in the presence of an acidic compound of food quality, wheat starch or corn starch, and which is in the form of soluble fibers. These fibers are introduced in the process object of the invention in powder form. It is, for example, possible to find this compound sold under the name Nutriose® FB by the company Roquette.

The polyols can be chosen, in a nonlimiting manner, from maltitol, mannitol, sorbitol, xylitol or isomalt.

The polyol is preferably maltitol, for example sold by the company Roquette under the brand Lycasin®.

According to another preferred embodiment, the polyol can be sorbitol. The sorbitol can be introduced in the process object of the present invention in powder form and can also exhibit good humectant and stabilizing properties in addition to its sweetening power. It is found, for example, sold under the name Neosorb® by the company Roquette.

When the polyol is introduced in powder form, sugar syrup is preferably introduced with the polyol so as to obtain the final syrupy product.

The amount of dextrin-based soluble fibers or of polyols can in particular be between 5% and 20% relative to the total weight of the final syrupy product obtained. Preferably, the amount of dextrin-based soluble fibers or of polyols is between 7% and 16% relative to the total weight of the final syrupy product obtained.

The introduction of the sugar syrup and/or sugar derivative and vitamins is carried out under an inert atmosphere in order to limit the amount of dissolved oxygen in the mixture comprising the sugar syrup and the vitamins.

For the purposes of the present invention, the expression “introduction under an inert atmosphere” is intended to mean that the equipment, in particular the tank into which the constituents of the syrupy product are introduced, is equipped with an inlet for inert gas at the bottom of the tank, by means, for example, of a sintered stainless steel dip pipe, which makes it possible to bubble inert gas into the content of the tank.

According to one preferred embodiment, the bubbling can in particular be carried out with a flow rate ranging from 0.1 to 10 Nm³/h, preferably from 2 to 8 Nm³/h, and more preferentially still of about 4.5 Nm³/h.

According to another preferred embodiment, the bubbling can in particular be carried out with a pressure ranging from 0.1 to 1 bar, preferably from 0.3 to 0.7 bar and more preferentially still of about 0.45 bar.

First of all, the sugar syrup and/or sugar derivative is (are) introduced into the main preparation tank.

According to one preferred embodiment, the sugar syrup is introduced via the top of the tank and the sugar derivative is introduced by suction at the bottom of the tank. In the latter case, the tank is advantageously equipped with a vacuum pump which makes it possible to create a vacuum in the tank and to suction the sugar derivative.

In order to further reduce the dissolved oxygen content in the sugar syrup and/or the sugar derivative, it is preferable to maintain the sugar and/or sugar derivative under stirring and bubbling of an inert gas for a period of time ranging from 1 to 6 hours, preferably from 2 to 5 h and more preferentially of 4 h.

According to one preferred embodiment, the sugar syrup and/or sugar derivative can be heated at a temperature which in particular does not exceed 50° C. This is because, at a temperature in excess of 50° C., the fat-soluble vitamins will undergo immediate degradation when they are introduced into the sugar syrup and/or sugar derivative.

The pH of the sugar syrup and/or sugar derivative can preferably be adjusted to between 8 and 10, more preferentially between 8.5 and 9.5, and in particular to around 9, for example by introducing a base such as sodium hydroxide. The adjustment of the pH makes it possible, on the one hand, to stabilize and protect the intermediate syrupy product formed at this stage, but also to preserve the fat-soluble vitamins introduced into the sugar syrup and/or sugar derivative.

Indeed, these fat-soluble vitamins must be in a basic medium at the time of their introduction. The degradation of said vitamins is promoted if this is not the case.

The vitamins are therefore subsequently introduced into the sugar syrup and/or sugar derivative.

According to one preferred embodiment, the vitamins are introduced sequentially into the sugar syrup and/or sugar derivative, i.e. the fat-soluble vitamins are introduced first, and then the water-soluble vitamins are introduced.

According to another preferred embodiment, the fat-soluble vitamins are predispersed with a surfactant, such as, in particular, an oil-in-water surfactant, of chemical origin or natural origin, such as a monoglyceride, a diglyceride, a sucrose ester, a phospholipid, a polysaccharide, for instance galactomannan, pectin, casein, gum arabic or gelatin, before they are introduced into the sugar syrup and/or sugar derivative. This operation makes it possible to facilitate the emulsification of the fat-soluble vitamins with the sugar syrup. Indeed, the surfactant acts as an initiator in creating the overall vitamin emulsion.

It should be noted that this preparation will also promote the homogenization of the fat-soluble vitamins with the water-soluble vitamins, once the latter are added to the mixture.

As previously described, vitamins are sensitive to heat and limiting the heating temperature makes it possible to avoid degradation thereof during the process. Thus, heating of the pre-emulsion at a temperature below 50° C., but above 35° C., allows good homogenization of the mixture without degrading the fat-soluble vitamins.

In order not to lose any of the vitamins introduced, the tank containing the pre-emulsion can in particular be rinsed after said pre-emulsion has been transferred into the sugar syrup and/or sugar derivative. The rinsing can in particular be carried out with water, preferably purified and deoxygenated water, always at a temperature which does not exceed 50° C.

Once the mixture of sugar syrup and/or sugar derivative and fat-soluble vitamins is homogeneous, the water-soluble vitamins can be introduced.

The temperature of the mixture into which the water-soluble vitamins are introduced can also be reduced to a temperature which does not exceed 35° C. This low heating temperature makes it possible to not degrade the water-soluble vitamins which are sensitive to heat, in particular above 35° C.

In order not to lose vitamins, the reservoirs containing the vitamins can be rinsed, in particular with water, preferably with deoxygenated and purified water, and even heated, but at a temperature which does not exceed 35° C.

According to one very particular embodiment of the present invention, it is also possible to envision introducing the vitamins into the process in the form of a premix of the fat-soluble and water-soluble vitamins. This premix advantageously has all the properties of the sequential introduction of the vitamins, namely, in particular, an absence of degradation of the latter and an ability to produce a well-formed emulsion. This premix is sold by the company Vitablend, for example.

According to one particular embodiment, it is also possible to introduce a flavoring, such as, in particular, a plum flavoring, into the mixture comprising the sugar syrup and/or sugar derivative and vitamins, in order to improve the taste of the syrupy product comprising vitamins. The flavoring can in particular be introduced into the syrup at the same time as the water-soluble vitamins.

### Addition of Dehydrated and Deoxygenated Glucose

The process according to the invention can comprise a step c) of adding dehydrated glucose.

The dehydrated glucose can in particular be introduced into the tank containing the sugar syrup and/or sugar derivative and vitamins by means of a system for transferring the dehydrated sugar placed under constant deoxygenation, i.e. an apparatus capable of transferring the dehydrated glucose into the tank without introducing oxygen therein.

According to one preferred embodiment, the dehydrated glucose is deoxygenated by means of an apparatus comprising a vacuum pump, said apparatus subsequently making it possible to introduce the glucose into the tank by means of an overpressure of inert gas, such as, in particular, of nitrogen.

Such an apparatus is in particular described in patent U.S. Pat. No. 6,325,572 and is sold by the company Dietrich Engineering Consultants under the name PTS®.

This type of apparatus operates by creating a vacuum in the body of said transfer apparatus, and then filling it with the powder of interest, namely dehydrated and deoxygenated glucose. Finally, using the pressure differential between the inside of this apparatus placed under a nitrogen pressure and the tank of the present process to which it is attached, the dehydrated and deoxygenated glucose is released into the tank of said process.

### Deoxygenation of the Syrupy Product

Once the various constituents of the syrupy product prepared by means of the process according to the invention have been mixed, the process according to the invention comprises a step d) of deoxygenating the syrupy product thus formed, in order to obtain the lowest possible oxygen content, preferably free of oxygen, before the packaging step.

The step of deoxygenating the syrupy product can in particular be carried out in a static mixer, for example a static mixer sold under the trade name SMV® by the company Sulzer.

For the purposes of the present invention, the term “static mixer” is intended to mean a reservoir comprising inverted-blade impellers, into which an inert gas is injected, subjecting the liquid to be deoxygenated to turbulence.

These inverted-blade impellers make it possible to increase the time spent by said liquid in the static mixer and mix the liquid mixed with the inert gas, in a turbulent system. The greater the number of inverted-blade impellers, the more turbulent the system is and the more efficient the deoxygenation proves to be.

According to one preferred embodiment, the static mixer is operated with an inert gas flow rate ranging from 0.1 to 20 Nm³/h, preferably from 1 to 10 Nm³/h and more preferentially still of about 8.5 Nm³/h.

According to another preferred embodiment, the static mixer is operated with an inert gas pressure ranging from 0.1 to 12 bar, preferably from 0.2 to 5 bar and more preferentially still of about 0.45 bar.

According to another preferred embodiment, the static mixer has a length of 1 m and a diameter of 50 cm. Depending on the flow rate and the viscosity of the liquid injected into this mixer, the dimensions of the static mixer and the number of inverted-blade impellers can be modified accordingly.

On leaving the static mixer, the liquid is saturated with microbubbles of inert gas and no longer contains any dissolved oxygen.

The dissolved oxygen content can be verified using an oxygen probe as previously described.

The dissolved oxygen content in the syrupy product after the deoxygenating step can in particular be a value ranging from 0 to 0.4 ppm, preferably from 0 to 0.2 ppm and more preferentially still from 0 to 0.1 ppm.

### Packaging of the Syrupy Product

The final step of the process according to the invention is the packaging of the syrupy product in individual bottles.

According to one preferred embodiment, the syrupy product obtained by means of the process according to the present invention is packaged in bottles of “aerosol” type.

The syrupy product can be conveyed to the packaging bottles by means of a hopper, connected to the static mixer and to said bottles via pipes.

For the purposes of the present invention, the term “hopper” is intended to mean a cone-shaped, sealed funnel acting as a mechanical buffering reservoir, which makes it possible to adjust the flow rate of the liquid.

The volume of syrupy product introduced into each bottle can in particular be controlled by using a metering system, for instance a positive-displacement pump, a weighing system, an optical system or a time-pressure system.

The packaging step of the process according to the invention is carried out under an inert atmosphere. Thus, the two pipes around the hopper and the hopper itself are inerted before the syrupy product passes through.

In particular, the bottles in which the syrupy product is packaged are inerted before they are filled, by injection of an inert gas.

According to one preferred embodiment, the inert gas is injected with a pressure ranging from 0.1 to 5 bar, preferably from 0.5 to 2 bar and more preferentially still of about 1 bar.

According to another preferred embodiment, the inert gas is injected for a period of time ranging from 10 to 2000 milliseconds, preferably from 50 to 500 milliseconds and more preferentially still of about 100 milliseconds.

It should be noted that the process according to the invention allows optimum preservation of the syrupy product. In particular, when the product is packaged in the form of aerosols, optimum preservation is maintained even when the product has already been opened for the first time. This is because the aerosol form of said bottle combined with the presence of nitrogen found in the aerial part of the bottle allow preservation of the syrupy product even when the user has already begun to consume a part of said syrupy product contained in said bottle. This type of bottle can be referred to as a pressurized bottle.

At this step, what the applicant will call the individual vitamin ratio and the overall vitamin ratio can be calculated.

According to one preferred embodiment, the individual vitamin ratio can in particular be greater than 95%, preferably greater than 98% and more preferentially greater than 98.6%.

According to a more preferred embodiment, the overall vitamin ratio can in particular be greater than 98%, preferably greater than 99% and more preferentially greater than or equal to 99.3%. In particular, those skilled in the art will take care to select vitamins which have individual vitamin ratios sufficiently high to enable the desired overall vitamin ratio to be obtained.

### Intermediate Storage of the Syrupy Product

Between the step of deoxygenating the syrupy product and the packaging step, it is possible to envision an intermediate step of storing the syrupy product in a tank.

In order to keep the dissolved oxygen content in the syrupy product to a minimum, the intermediate step of storing the syrupy product in a tank is carried out under an inert atmosphere and the syrupy product is kept stirring.

In particular, the storage tank can be inerted before the transfer of the syrupy product into said tank. Once the syrupy product has been transferred into the storage tank, said product can be kept under an inert atmosphere by bubbling of an inert gas.

According to one preferred embodiment, the bubbling is carried out with a flow rate ranging from 0.1 to 20 Nm³/h, preferably from 1 to 10 Nm³/h and more preferentially still of about 2 Nm³/h.

According to another preferred embodiment, the bubbling is carried out with a pressure ranging from 0.1 to 12 bar, preferably from 0.2 to 5 bar and more preferentially still of about 0.45 bar.

According to another embodiment of the invention, the latter can consist of a pressurized bottle comprising a syrupy product directly obtainable by means of the process object of the invention.

According to this other embodiment of the invention, the pressurized bottle may be of aerosol type, comprising a syrupy product directly obtainable by means of the process object of the present invention and characterized in that the overall vitamin ratio of said syrupy product is greater than or equal to 99.3%.

The invention is illustrated in greater detail by means of the following examples which are given purely by way of nonlimiting illustration.

### EXAMPLES

### Example 1

### Elements of the Process Carried Out

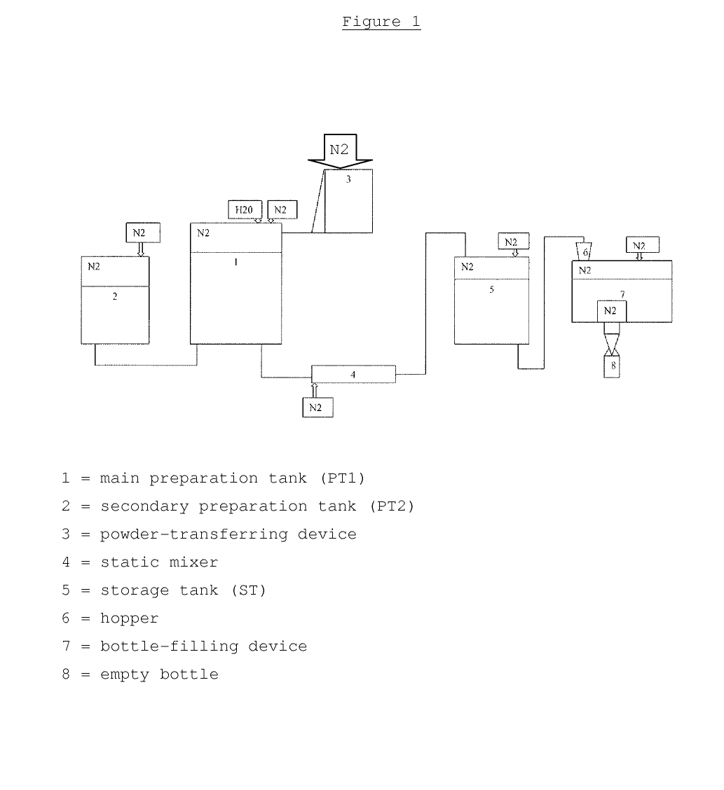

A process according to FIG. 1 is carried out, said process comprising the following elements: a main preparation tank (PT1) and a secondary preparation tank (PT2) which are connected via a pipe. These two preparation tanks are used for mixing the various constituents of the syrupy product. These two tanks are heated with double jackets in which, a heat-exchange liquid (of the water or oil type) circulates within the confined space. The two preparation tanks are each equipped with an inlet for inert gas in order to perform the inerting of the tanks and with a sintered stainless steel pipe at the bottom of the tank, which makes it possible to bubble inert gas into the content of the tanks. The two preparation tanks are also equipped with stirring spindles, of the impeller blade or else turbine type, in order to be able to mix the content of the tanks. The main preparation tank is also fitted with a powder-transferring apparatus sold by the company Dietrich Engineering Consultants in order to introduce the dehydrated glucose into the tank without causing oxygen to enter therein. The main preparation tank also comprises a water inlet, the water being purified and deoxygenated beforehand by means of a static exchanger. The main preparation tank is connected to a static mixer via a pipe. The static mixer is sold under the trade name SMV® by the company Sulzer; it has a length of 1 m and a diameter of 50 cm. It is fitted with an inlet for inert gas. The static mixer is connected to a storage tank (ST) via a pipe. The storage tank is fitted with an inlet for inert gas in order to inert the tank and with a sintered stainless steel pipe at the bottom of the tank which makes it possible to bubble inert gas into the content of the tank. The storage tank is also equipped with a turbine or an impeller blade, in order to be able to mix the content of the tank. The storage tank is connected to the packaging station via a pipe. The packaging station consists of a hopper and a bottle-filling machine, which are connected to one another via a pipe. The metering system, namely a positive-displacement pump, makes it possible to adjust the flow rate of the liquid which reaches the bottle-filling machine. The bottle-filling machine is fitted with a nozzle which fits the necks of the bottles and can thus inert them, fill them with the syrupy product comprising vitamins and keep them under an inert atmosphere until they are stoppered, but also after opening of the consumable, in particular during daily use by the consumer.

### Example 2

### Preparation of a Syrupy Product by Means of the Process According to the Invention

In the process for preparing the syrupy product according to the invention, the following amounts of raw materials are used:

| CONSTITUENTS | AMOUNTS (in kg) |
|---|---|
| Sugar syrup | 2000 |
| Sodium hydroxide | 12.5 |
| Ascorbic acid | 3 |
| Montanox 80 | 12.2 |
| Vitamin A | 0.350 |
| Vitamin E | 3.6 |
| Flavoring | 0.2 |
| Vitamin B6 | 0.35 |
| Vitamin B3 | 3.2 |
| Vitamin B2 | 0.360 |
| Vitamin B5 | 1.1 |
| Dehydrated glucose | 184 |
| Purified water | qs 2600 |

The process can be broken down into the following steps:
the two preparation tanks (PT1 and PT2) and the pipe connecting them are inerted with an overpressure of inert gas at 0.4 bar for 300 sec.;
the sugar syrup is introduced into the main preparation tank (PT1);
the sugar syrup is left to stir with bubbling of nitrogen with a pressure of 0.4 bar and a flow rate of 4 Nm³/h for 4 hours;
the sugar syrup is heated at 50° C. by means of the double jackets surrounding tank PT1;
the pH of the sugar syrup is adjusted to 9.5 by introducing the sodium hydroxide and the ascorbic acid;
the secondary preparation tank (PT2) is preheated at 50° C. by means of the double jackets surrounding tank PT2;
the Montanox 80 and the following fat-soluble vitamins: vitamin A, vitamin D3, vitamin E, are introduced into the secondary preparation tank (PT2);
the mixture in tank PT2 is left to stir at 50° C. and with bubbling of nitrogen for 60 minutes;
the content of tank PT2 is transferred to tank PT1 via the pipe connecting them;
tank PT2 is rinsed several times with water, at 50° C., purified and deoxygenated beforehand, and the rinsing water is then transferred to tank PT1 via the pipe connecting PT2 to PT1;
the content of tank PT1 is left to stir at 50° C. and with bubbling of nitrogen for 60 minutes;
the temperature of the content of tank PT1 is reduced to 35° C.;
the following water-soluble vitamins: vitamin B1, vitamin B2, vitamin B3, vitamin B5, vitamin B6, vitamin B8, vitamin B12 and vitamin C, and also the plum flavoring, are introduced into tank PT1;
the reservoirs which contained the vitamins are rinsed with purified water and the rinsing water is added to tank PT1;
the content of tank PT1 is left to stir at 35° C. and with bubbling of nitrogen for 120 minutes;
the dehydrated glucose is introduced into tank PT1 via the powder-transferring system;
purified and deoxygenated water is introduced into tank PT1;
the content of tank PT1 is left to stir at 35° C. and with bubbling of nitrogen for 60 minutes;
the piping between tank PT1 and the storage tank is inerted, and the storage tank is inerted with a nitrogen pressure at 0.4 bar and a flow rate of 1 Nm³/h for 300 seconds;
the content of tank PT1 is transferred to the storage tank passing through the mixer into which nitrogen is injected, in contraflow to the passage of the liquid, with a pressure of 0.4 bar and a flow rate of 8 Nm³/h;
the syrupy product which gradually accumulates in the storage tank is kept stirring at 150 rpm and with bubbling of nitrogen with a pressure of 0.4 bar and a flow rate of 2 Nm³/h;
the pipes between the storage tank and the bottle-filling machine, and also the packaging hopper, are inerted with nitrogen at a pressure of 0.3 bar for 600 seconds;
the syrupy product is transferred to the packaging hopper and the content is kept at a nitrogen pressure of 0.2 bar;
the syrupy product is gradually transferred to the bottle-filling machine;
the bottles are inerted with nitrogen at 1 bar for 100 milliseconds, then they are filled with a volume of syrupy product metered by a positive-displacement pump and they are kept under a nitrogen atmosphere until they are closed by means of a crimped valve.

One of the bottles produced is taken randomly and the vitamins contained in this bottle are assayed. The yields are calculated by dividing the actual amount of vitamins that is contained in the bottle by the theoretical amount of vitamins that should be in the bottle, and by multiplying the result by 100. The yields are collated in the following table:

| VITAMINS | YIELD |
|---|---|
| Vitamin A | 100% |
| Vitamin B2 | 98.6% |
| Vitamin B3 | 98.8% |
| Vitamin B5 | 100% |
| Vitamin B6 | 98.6% |
| Vitamin E | 100% |
| All the vitamins | 99.3% |

It is noted that the various individual vitamin ratios are very good since they are between 98.6% and 100%.

The same is true for the overall vitamin ratio, since it is 99.3%.

Thus, the specifications indicated on the bottles for each vitamin are observed without having to overdose the vitamins, making it possible to thus obtain a syrupy vitamin product of pharmaceutical quality.

It should be noted that this particular embodiment of the process, which is the subject of the present invention, is in no way limiting with respect to the raw materials exemplified above that can be introduced during the preparation of said syrupy product. Thus, any product which can be ingested by human beings, that is sensitive to oxygen, or else which can be in the form of an emulsion, can be integrated into said manufacturing of said syrupy product, without being limited to a defined amount. For example, it is possible to envision introducing, in addition, various compounds chosen from the nonlimiting list comprising probiotics, mineral salts, hormones, amino acids, certain active ingredients derived from plants, alkaloids, preservatives such as potassium sorbate or else antioxidants. Of course, the introduction of these various compounds has no effect on the quality of the syrupy product obtained.

### Example 3

### Preparation of a Syrupy Product by Means of the Process According to the Invention

According to one embodiment similar to the process described in example 2, a syrupy product comprising the following amounts of raw materials is prepared:

| CONSTITUENTS | AMOUNTS (in kg) |
|---|---|
| Sugar syrup | 2000 |
| Sodium hydroxide | 12.5 |
| Ascorbic acid | 3 |
| Montanox 80 | 12.2 |
| Vitamin C | 16 |
| Vitamin D3 | 0.001 |
| Vitamin A | 0.350 |
| Vitamin E | 3.6 |
| Flavoring | 0.2 |
| Vitamin B1 | 0.247 |
| Vitamin B8 | 0.01 |
| Vitamin B12 | 0.55 |
| Vitamin B6 | 0.35 |
| Vitamin B3 | 3.2 |
| Vitamin B2 | 0.360 |
| Vitamin B5 | 1.1 |
| Dehydrated glucose | 184 |
| Purified water | qs 2600 |

In accordance with the last paragraph of example 2, it is thus possible to prepare an entire variety of syrupy products of which the raw materials used and also the amounts thereof introduced during the implementation of said process are entirely variable. On the other hand, the quality of the syrupy product obtained at the end of the process remains the same.

### Example 4

### Preparation of a Syrupy Product by Means of the Process According to the Invention

According to one particular embodiment of the invention, a syrupy product comprising the following amounts of raw materials is prepared:

| CONSTITUENTS | AMOUNTS (in kg) |
|---|---|
| Sugar syrup | 2000 |
| Sodium hydroxide | 12.5 |
| Ascorbic acid | 3 |
| Montanox 80 | 12.2 |
| Vitamin C | 16 |
| Vitamin D3 | 0.001 |
| Vitamin A | 0.350 |
| Vitamin E | 3.6 |
| Flavoring | 0.2 |
| Vitamin B1 | 0.247 |
| Vitamin B8 | 0.01 |
| Vitamin B9 | 0.06 |
| Vitamin B6 | 0.35 |
| Vitamin B3 | 3.2 |
| Vitamin B2 | 0.360 |
| Vitamin B5 | 1.1 |
| Potassium sorbate | 5.09 |
| Maltitol syrup | 409 |
| Purified water | qs 2600 |

In accordance with the last paragraph of example 2, it is thus possible to prepare an entire variety of syrupy products of which the raw materials used and also the amounts thereof introduced during the implementation of said process are entirely variable. On the other hand, the quality of the syrupy product obtained at the end of the process remains the same.

1. A process for preparing a syrupy product comprising vitamins, said process comprising:
a) inerting all the equipment used in the process;
b) introducing, into said equipment, a sugar syrup and/or a sugar derivative and introducing the vitamins under an inert atmosphere;
c) deoxygenating the syrupy product thus formed;
d) packaging the syrupy product under an inert atmosphere.

2-15. (canceled)

16. The process according to claim 1 further comprising an additional step of adding dehydrated and deoxygenated glucose under an inert atmosphere after step b).

17. The process according to claim 1, wherein the sugar derivative is dextrin-based soluble fibers or polyols.

18. The process according to claim 1, wherein introduction of the vitamins under an inert atmosphere is carried out sequentially.

19. The process according to claim 1, wherein the step of introducing the vitamins also comprises introducing a surfactant of chemical or natural origin chosen from the list of gelatins, pectins, casein, gum arabics or galactomannans, and also water purified and deoxygenated beforehand.

20. The process according to claim 1, further comprising an intermediate step of storing the syrupy product in a tank under an inert atmosphere and with stirring between the steps of deoxygenating and of packaging the syrupy product.

21. The process according to claim 1, wherein the inerting of the atmosphere is carried out in the equipment used in the process before the introduction of any raw material, and is maintained by bubbling an inert gas with a flow rate ranging from 0.1 to 20 Nm³/h, and a pressure ranging from 0.1 to 1 bar.

22. The process according to claim 1, wherein the dehydrated glucose is deoxygenated by means of a system for transferring dehydrated sugar placed under constant deoxygenation, said system subsequently making it possible to introduce the glucose into the tank.

23. The process according to claim 1, wherein the step of deoxygenating the syrupy product is carried out in a static mixer operated with an inert gas flow rate ranging from 0.1 to 20 Nm³/h, and a pressure ranging from 0.1 to 12 bar.

24. The process according to claim 1, wherein the dissolved oxygen content in the syrupy product after the deoxygenating step is a value ranging from 0 to 0.4 ppm.

25. The process according to claim 1, wherein the packaging of the syrupy product is carried out by means of a metering system.

26. The process according to claim 1, wherein the bottles in which the syrupy product is packaged are inerted before they are filled, by injection of an inert gas with a pressure ranging from 0.1 to 5 bar, for a period of time of between 10 and 2000 milliseconds.

27. The process according to claim 1, wherein the inert gas is nitrogen.

28. The process according to claim 1, wherein the individual vitamin ratio is greater than or equal to 98.6%.

29. The process according to claim 1, wherein the overall vitamin ratio is greater than or equal to 99.3%.

30. A pressurized bottle comprising a syrupy product directly obtainable by means of the process as defined in claim 1.

31. The pressurized bottle comprising a syrupy product according to claim 30 wherein the overall vitamin ratio of said syrupy product is greater than or equal to 99.3%.
